(19) United States
(12) Patent Application Publication
(10) Pub. No.: US 2025/0354170 A1
(43) Pub. Date: Nov. 20, 2025

(54) METHOD FOR THE MANUFACTURE OF RECOMBINANT ADENOVIRUS VECTORS

(71) Applicant: Ferring International Center S.A., Saint-Prex (CH)

(72) Inventor: Minna Hassinen, Kuopio (FI)

(21) Appl. No.: 19/209,918

(22) Filed: May 16, 2025

Related U.S. Application Data
(60) Provisional application No. 63/648,224, filed on May 16, 2024.

(30) Foreign Application Priority Data
May 16, 2024 (EP) 24305765.0

(51) Int. Cl. C12N 15/86; C12N 7/00

(52) U.S. Cl. CPC C12N 15/86; C12N 7/00; C12N 2710/10043; C12N 2710/10052

(57) ABSTRACT

The present disclosure relates to a method for producing a recombinant adenovirus vector.

### CROSS REFERENCE TO RELATED APPLICATIONS

This application claims priority to U.S. Provisional Application No. 63/648,224, filed May 16, 2024, and EP application Ser. No. 24/305,765.0, filed May 16, 2024, each of which are hereby incorporated by reference in their entirety.

### FIELD OF THE DISCLOSURE

The present disclosure relates to a method for producing a recombinant adenovirus vector.

### BACKGROUND OF THE DISCLOSURE

Recombinant adenovirus vectors have emerged as a potent therapeutic means to treat a number of diseases, including cancers. For example, nadofaragene firadenovec (otherwise referred to as Adstiladrin), is a gene therapy product approved by the U.S. Food and Drug Administration for the treatment of adult patients with high-risk Bacillus Calmette-Guérin (BCG)-unresponsive non-muscle invasive bladder cancer (NMIBC) with carcinoma in situ (CIS) with or without papillary tumors. Such gene therapy vector has proven to be effective to treat subjects in need thereof. Scale-up of the production of high-quality vectors is needed for accelerating patient access to these innovative therapies. The present disclosure provides improved methods for the production of recombinant adenovirus vectors, at scales compatible with the clinical demand.

### SUMMARY OF THE DISCLOSURE

As mentioned above, the present disclosure provides methods for the production of recombinant adenovirus vectors. Implementation of the methods disclosed herein results in the high scale manufacturing of such vectors for therapy.

More specifically, the disclosure relates to a method for producing a recombinant adenovirus vector, comprising the following steps:
a) inoculating cells into a bioreactor comprising a carrier providing a surface area for adherent cell culture;
b) infecting said inoculated cells with said recombinant adenovirus vector;
c) lysing the cells into the bioreactor;
d) performing a harvest step of recombinant adenovirus vectors from said lysed cells thereby obtaining a bulk harvest of recombinant adenovirus vectors; and
e) filtering said bulk harvest thereby obtaining a filtered bulk harvest.

### DETAILED DESCRIPTION

It is herein described effective methods for the production of recombinant adenovirus vectors. This streamlined method achieves high yields of high-quality vectors of clinical grade, that can be administered to a mammalian subject, such as a human subject in need thereof.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. Likewise, the term “include” and its grammatical variants are intended to be non-limiting, such that recitation of items in a list is not to the exclusion of other like items that can be substituted or added to the listed items. The present disclosure contemplates other embodiments “comprising,” “consisting of” and “consisting essentially of,” the embodiments or elements presented herein, whether explicitly set forth or not.

The singular forms “a,” “and” and “the” include plural references unless the context clearly dictates otherwise. Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Units, prefixes, and symbols are denoted in their Système international d′unités (SI) accepted form. Unless otherwise indicated, nucleic acids are written left to right in 5′ to 3′ orientation; amino acid sequences are written left to right in amino to carboxy orientation.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### 2. Recombinant Adenovirus Vectors

The disclosure relates to the production of recombinant adenovirus vectors. The recombinant adenovirus vectors described herein are suitable to deliver a nucleic acid of interest to a cell of a mammalian subject and express the product encoded by said nucleic acid of interest into or from said cell.

In a particular embodiment, the recombinant adenovirus vector may be replication-competent (e.g., meaning that the virus is capable of infecting cells and replicating to produce additional infectious virus) or replication-defective, production of replication-defective adenovirus vectors being preferred. In yet another embodiment, it is herein described the production of recombinant adenovirus serotype 5 (rAd5) vectors, in particular replication-defective rAd5 vectors.

In a particular embodiment, the nucleic acid of interest encodes a therapeutic product and thus the recombinant adenovirus vector is useful for gene therapy. The therapeutic product may be a therapeutic protein or a therapeutic RNA (e.g. a siRNA, miRNA or shRNA), in particular a therapeutic protein. In a further particular embodiment, the therapeutic protein is suitable to treat cancer, in particular bladder cancer, such as non-muscle invasive bladder cancer (NMIBC) or muscle invasive bladder cancer (MIBC). In a further embodiment, the therapeutic protein is human interferon, such as a human Type 1 or Type 2 interferon, in particular human Type 1 interferon, such as human interferon α or β, in particular human interferon a, more particularly interferon α2, even more particularly human interferon α2b.

In addition to said nucleic acid of interest, the genome of the recombinant adenovirus vector 5 comprises one or more regulatory elements suitable to effectively express the nucleic acid of interest in or from the target cell. Such regulatory elements include, without limitation, promoters, enhancers, silencers and/or insulators.

According to a particular embodiment, the recombinant adenovirus vector is a replication-defective rAd5 vector encoding human interferon α2b. Said vector may be constructed with the human gene in an expression cassette which replaces the E1a, E1b and pIX regions at the 5′ end of the adenovirus genome, as is known in the art. Such vector construction can be performed by the skilled person, using standard DNA manipulation techniques.

In a further particular embodiment, the replication-defective rAd5 vector encoding human interferon α2b is nadofaragene firadenovec.

### 3. Method for the Production of Recombinant Adenovirus Vectors

Production of recombinant adenovirus vectors have been described previously, for example in WO2016/048556. The present disclosure provides an unexpected improvement over that previously described method.

The method for producing a recombinant adenovirus vector according to the present disclosure comprises the following steps:
a) inoculating cells into a bioreactor comprising a carrier providing a surface area for adherent cell culture;
b) infecting said inoculated cells with said recombinant adenovirus vector;
c) lysing the cells into the bioreactor;
d) performing a harvest step of recombinant adenovirus vectors from said lysed cells thereby obtaining a bulk harvest of recombinant adenovirus vectors; and
e) filtering said bulk harvest thereby obtaining a filtered bulk harvest of recombinant adenovirus vectors.

### Producer Cells

In the methods of the present disclosure, in step a, inoculated cells are used as producer cells. As will be apparent herein, production of the recombinant adenovirus vector is carried out after infection of the cells with the recombinant adenovirus vector. Thus, the cells used in the present disclosure are permissive to infection by said recombinant adenovirus vector and contain the elements required to propagate the recombinant adenovirus vector after infection. A suitable producer cell is selected depending on the desired recombinant adenovirus vector to be produced. Many therapeutic adenoviral vectors, such as rAd5 vectors, are non-replicating (or replication deficient) whereby the genome is deleted in the E1 region to provide space for alternate gene expression cassettes. The E1 region encodes proteins necessary for the expression of the other early and late genes, hence initiating the viral life cycle. Therefore, when the E1 region is replaced with an expression cassette to produce the gene product that is useful in therapy, such as cytokines, suicide genes, antigens and antibodies, a producer cell line containing adenovirus E1 sequences is required to complement for this region. Therefore, in a particular embodiment in which the recombinant adenovirus vector lacks the adenovirus E1 region, the producer cell used in the practice of the present disclosure contains adenovirus E1 sequences. For example, the cells that can be used in the present disclosure can be selected from HEK293, 293T, or any HEK293 related cell line, Per.C6 or SL0036 cells. In a particular embodiment, HEK293 or HEK293T cells are used, in particular HEK293 cells.

### Expansion of Cells Before Inoculation

According to the present disclosure, the cells are grown in culture in adherent mode into the bioreactor for production of the recombinant adenovirus vector. Before inoculation into the bioreactor, the cells may be expanded in adherent mode or in suspension. In a particular embodiment before inoculation into the bioreactor, the cells may be expanded as a non-adherent, suspension culture of suspension-adapted cells, as described in WO2016/048556. Expansion of cells in adherent mode may be conducted in a medium comprising serum or devoid of serum, in particular comprising serum. In a particular embodiment, expansion of cells in suspension is conducted in a substantially serum-free medium, such as a serum-free defined cell culture medium, which is devoid of animal-derived serum. A defined medium contains components whose exact concentration is known, such as a basal medium (e.g. DMEM, F12 or RPMI), amino acids, vitamins, inorganic salts, buffers, antioxidants and energy source (such as glucose, L-glutamine and/or L-alanyl-L-glutamine). It may be further supplemented with recombinant proteins or peptides, such as recombinant albumin, recombinant insulin. Such defined media are commercially available. For the culture of HEK293 cells, suitable media include, without limitation, CD 293 medium, BHK, Ex-Cell medium, Freestyle or Hyclone SFM293 culture media may be used. According to a particular embodiment, Ex-Cell medium can be used during cell expansion. Starting from an initial batch of cells, such as from a working cell bank (WCB), depending on the quantity of cells to be inoculated into the bioreactor implemented in step a) of the method of the present disclosure, and of the size of the bioreactor, a number of cycles of expansion can be implemented. An illustrative, non-limiting expansion process is provided in the following part of this paragraph. After thawing of cells from a WCB vial, expansion may be started in suspension using one or more culture vessels, such as one or more flasks, for example T-flask(s) or roller bottle(s), which can be followed by a proliferation period in other flasks or roller bottles until a sufficient number of cells is reached to further expand the cells. In a particular embodiment, once a quantity of about 1×10⁸ to about 1×10¹⁰ cells is reached, further expansion can be implemented in a larger scale system, such as in a batch, fed-batch or perfusion system until a sufficient number of cells is reached for inoculation into the bioreactor. In a particular embodiment, further expansion is conducted until a total of about 1.0×10⁹ to about 1.0×10¹¹ cells is reached. Target cell density may vary from about 1×10⁵ to about 1×10⁷ cells/mL, in particular, from about 0.5×10⁶ to about 0.5×10⁷ cells/mL. In a particular embodiment, such target cell density is obtained by inoculation of about 1.0×10⁹ to about 1.0×10¹¹ cells into a perfusion bioreactor. In a particular embodiment, culture in the perfusion bioreactor starts at a volume of about 5 L of culture medium. The cultivation volume may be increased to reach a final working volume, such as a volume of about 10 L, within a suitable period of time, such as within 1 to 3 days after inoculation, for example within about two days after inoculation of the perfusion bioreactor. 16 to 48 hours reaching the final working volume, for example after about 24 hours, perfusion may be started to enhance cell proliferation to reach the target cell density.

### Inoculation of the Bioreactor

Expanded cells can then be used for inoculation of the bioreactor in step a) of the production method.

As mentioned above, the cells are grown in adherent mode in the bioreactor. In order to increase adherent surface, the bioreactor may contain a carrier onto which expanded cells can anchor. Such carrier may be either floating or fixed in the bioreactor. In a particular embodiment, the bioreactor is a fixed bed bioreactor. Illustrative carriers for practice of the disclosure include, without limitation, fiber carriers, microcarriers (e.g. beads) and macrocarriers, in particular fiber carriers such as packed-fiber carrier. Such carriers can be made, for example, using polyethylene terephthalate, polystyrene, polyester, polypropylene, DEAE-dextran, collagen, glass, alginate or acrylamide. Suitable bioreactors which may be used include those containing bead-type micro-carriers (e.g., Cytodex® brand beads, commercially available from Cytiva) and matrix type carriers (e.g., Fibra-cell™ brand disks, commercially available from Eppendorf Corp.). According to a particular embodiment, the bioreactor comprises a polyester packed-fiber carrier such as that used in the iCELLis® 500 or iCELLis® 500+ bioreactors, (commercially available from Advanced Technology Materials Inc. (Brussels, Belgium) and Cytiva). In a further particular embodiment, the bioreactor comprises a carrier providing a surface area for cell growth comprised between about 66 m² and about 500 m², in particular between 100 m² and 500 m². In a further particular embodiment, the bioreactor comprises a polyester packed-fiber carrier such as that used in the iCELLis® 500 or iCELLis® 500+ bioreactors providing a surface area for cell growth comprised between about 66 m² and about 500 m². In a further particular embodiment, the bioreactor comprises a polyester packed-fiber carrier of about 500 m².

The medium used for growth into the bioreactor may comprise a defined medium, containing components whose exact concentration is known, such as a basal medium basal medium (e.g. DMEM, F12 or RPMI medium). The medium further contains factors suitable to promote cell adherence to the bioreactor or carrier contained therein. Such factors promoting adherence include, without limitation, cell adhesion factors, such as components of the extracellular matrix (e.g. fibronectin, collagen, laminin, calcium ions, or proteoglycans or non-proteoglycan polysaccharides of the extracellular matrix) and animal-derived serums, such as mammal derived serum, in particular fetal bovine serum (FBS). In a particular embodiment, the medium comprises animal-derived serum, in particular FBS. Such factors suitable to promote cell adherence may be added to the culture medium just before, during or after the inoculation of the suspension cells into the bioreactor. Suitable media include, without limitation, DMEM, F12 or RPMI medium, in particular DMEM, supplemented with FBS, for example FBS at a concentration between about 2 and about 20% v/v of the medium, in particular between about 5 and about 15%, e.g., about 9%. The medium may further comprise other components such as L-glutamine (for example at a concentration of about 0.5-about 5 mM, such as about 2 mM) or L-alanyl-L-glutamine (for example at a concentration of about 0.5-about 5 mM, such as about 2 mM).

Inoculation of the bioreactor with an effective quantity of suspension-expanded cells is performed, with said quantity varying depending on the bioreactor to be used. In certain non-limiting embodiments, inoculation may be performed to achieve a cell density of about 1000-about 40,000 cells/cm², in particular of about 2,000-about 20,000 cells/cm², more particularly of about 6,000 to about 15,000 cells/cm², more particularly of about 7,000-about 12,000 cells/cm², in particular of about 8,000-about 10,000 cells/cm². By way of an example, a bioreactor providing a surface area of about 100 m², such as the iCELLis® 500+/100 m² bioreactor, is inoculated in a particular embodiment with between about 1.0×10⁹ and about 1.0×10¹¹ cells, in particular between about 0.6 and about 1.5×10¹⁰ cells, more particularly between about 0.8 and about 1.0×10¹⁰ cells. By way of another example, a bioreactor can be used that provides a surface area of about 500 m², such as the iCELLis® 500+/500 m² bioreactor, in which in a particular embodiment, between about 5.0×10⁹ and about 1.0×10¹¹ cells, in particular between about 2.0 and about 7.0×10¹⁰ cells, more particularly between about 3.0 and about 6.0×10¹⁰ cells, in particular between about 4.0 and about 5.0×10¹⁰ cells are inoculated into the bioreactor. Before inoculation, the cells may be centrifuged to obtain a cell pellet, and suspended in an appropriate volume of growth medium. It is also possible to add cells into the bioreactor directly from suspension culture. For example, cells grown in suspension in Ex-Cell medium during cell expansion can be inoculated directly into the bioreactor containing, for example, DMEM with or without FBS. In this aspect, the small quantity of suspension medium will not affect the cell anchorage. Accordingly, cells in suspension culture can be transferred to the adherent culture bioreactor by any suitable means, including direct transfer, or by indirect transfer.

Culture volume may vary to a large extent, depending on the cells and type of bioreactor used 30 for the production of the recombinant adenovirus vector. In a particular embodiment, in particular when the cells are HEK293 cells or cells derived therefrom, the bioreactor can comprise (a) a polyester packed-fiber carrier providing a surface area for cell growth comprising between about 66 m² and about 500 m², and (b) from about 40 to about 100 L of culture medium, in particular from about 50 to about 80 L, such as from about 60 to about 70 L of culture medium, and in particular about 65 L of culture medium.

Cells may then be expanded for a period of time allowing reaching a cell density suitable for production of clinical or commercial batches of recombinant adenovirus vectors. In a particular embodiment, cells are expanded in the bioreactor for about 70 to about 150 hours before infection.

### Infection

In step c), inoculated cells are infected with the recombinant adenovirus vector to be propagated. In certain embodiments, infection of the cells may be performed at a multiplicity of infection (MOI) of about 110 to about 190 vp/cell, in particular of about 130 to 175 vp/cell, in particular of about 150 to about 170 vp/cell. In certain other embodiments, infection of the cells may be performed at a MOI of about 7 to 24 infectious particles/cell, in particular, about 9 to 21 infectious particles/cell.

One needs to determine the cell count in the bioreactor in order to determine the number of viral particles required to achieve the target MOI. However, counting of cells may not always be possible, depending on the type of bioreactor used, as is the case with a packed-fiber carrier made of polyester providing a surface area for adherent cell growth. In that case, the number of cells in the bioreactor can be theoretically determined based on the seeding density, expansion time between inoculation and infection, and doubling time of the cells used in the production. Determination of these parameters is routine practice for those skilled in the art. For example, with cells with a doubling time of about 24 hours, and inoculation at a cell density of about 10,000 cells/cm², after about 100 hours cultivation the cell density would be of about 180,000 cells/cm². As a further illustration, with cells with a doubling time of about 29 hours, and inoculation at a cell density of about 10,000 cells/cm², after about 110 hours cultivation the cell density would be of about 140,000 cells/cm².

In a particular embodiment, the cells, such as HEK293 cells or HEK293 related cells, more particularly HEK293 cells, are infected at a cell density of about 80,000 to about 150,000 cells/cm².

Duration of infection may vary from about 30 to about 80 hours, such as from about 50 to about 60 hours.

In a particular embodiment, the cell culture is carried out in batch or in perfusion mode. In certain embodiments, the cell culture is carried out in perfusion mode. In such embodiment, perfusion may be stopped just before infection, and the required volume of virus is added to the bioreactor to infect the expanded cells. Perfusion may be restarted about 30 to about 120 minutes after the start of the infection of the cells Moreover, in a further embodiment, approximately about 15 to about 50 hours post-infection, the perfusion media may be changed to serum-free media. This allows reducing the concentration of residual FBS at harvest. Perfusion may be continued until such harvest.

### Cell Lysis, Vector Harvest and Filtration

The formation of recombinant adenovirus vectors takes place inside the cells. Thus, release of such vectors may be carried out using a lysis step. Cell lysis may be carried out by any conventional physical or chemical means. In a particular embodiment, lysis is carried out inside the bioreactor by adding at least one detergent into to the bioreactor, such as a non-ionic surfactant. Examples of non-ionic surfactants that can be used as detergents include polysorbate, in particular polysorbate 20, or Triton-X. In a particular embodiment, the detergent is polysorbate, in particular polysorbate 20. In a particular embodiment, lysis is carried out with a lysis buffer comprising about 10% m/v polysorbate 20, such as about 10% m/v polysorbate. In a further particular embodiment, the spent media in the bioreactor is either kept or discarded, preferably discarded, before addition of the lysis buffer, and replaced by fresh serum-free medium, such as serum-free DMEM medium. Lysis is then continued for a sufficient time, such as for about 1 to about 3 hours, in particular for about 2 hours±about 10 minutes.

In a particular embodiment, endonuclease treatment is carried out to digest host cell nucleic acid molecules. The endonuclease treatment may be carried out any enzyme able to degrade DNA and/or RNA, preferably both. Such endonucleases include, without limitation, an endonuclease from Serratia marcescens, in particular a genetically engineered endonuclease from Serratia marcescens such as Benzonase®. In a further embodiment, endonuclease treatment is implemented as a two-step endonuclease treatment. Accordingly, a first endonuclease (e.g. Benzonase®) treatment is carried out before addition of the lysis buffer, and a second endonuclease (e.g. Benzonase®) treatment is carried out after addition of the lysis buffer. For example, in an illustrative, non-limiting embodiment, after replacement of spent media with fresh serum-free media, a first Benzonase® aliquot of approximately about 60 to about 100 U/mL, such as about 74 U/mL, is added to the bioreactor and incubated for about 25 to about 40 minutes, in particular for about 30 to about 35 minutes. Lysis buffer is then added and lysis is continued for the required time period. Then, a second Benzonase® aliquot of approximately about 300 to about 450 U/mL, such as, for example, about 369 U/mL, may be added to the bioreactor to digest host DNA for an additional about 25 to about 40 minutes, in particular for an addition about 30 to about 35 minutes. Benzonase may then be inactivated with a high concentration salt buffer, such as a buffer comprising about 3,500 to about 5,000 mM NaCl.

The recombinant adenovirus vectors can then be harvested by collecting the lysed material, thereby providing a main harvest material. In certain embodiments, the bioreactor may then further be rinsed with conditioning buffer to recover as many virus particles from the bioreactor as possible. Both the main harvest material and rinse may then be pooled. The main harvest, optionally pooled with the rinse, may be referred to as a bulk harvest.

According to the present disclosure, step e) of filtering said bulk harvest is then implemented to obtain a filtered bulk harvest. Such filtration may be carried out through an about 0.2 μm filter, such as about a 0.5/0.2 filter. According to a particular embodiment, the filter may be flushed with conditioning buffer. The resulting product is referred to as a filtered bulk harvest.

Purification of the released recombinant adenovirus vector can then be carried out.

### EXAMPLES

### Example 1. Thawing and Expansion of HEK293 Cells from Working Cell Bank (WCB)

The purpose of this process stage is to expand HEK293 cells from a WCB vial to produce enough cells for the inoculation of an adherent single-use bioreactor comprising an iCELLis 500+ carrier.

A vial of WCB were removed from liquid nitrogen and cells thawed by partial immersion of the vial in a water bath at +37° C. Cells were transferred to a 75 cm² cell culture flask and incubated in +37° C. and 5% CO2 for 3-4 days.

When expanding the cells in T-flasks and roller bottles, the cell number and viability was determined at each passage. Cell suspension was diluted with fresh cell culture medium. In a first expansion, cells were seeded to 1 to 2×75 cm² flasks and incubated at +37° C. and 5% CO₂ for 3-4 days. In the second expansion cells were seeded to 2 to 3×75 cm² flasks or one 850 cm² roller bottle, depending on the total number of cells and the total cultivation volume. Cells were cultivated at +37° C. and 5% CO₂ for 3-4 days.

In the third expansion, cells were expanded and transferred to 1 roller bottle within the volume. Cells were expanded further until the amount of 10 roller bottles (850 cm²) was achieved. Cells were grown in incubators (+37° C., 5% CO₂), on a roller device.

This cultivation process resulted in 10 roller bottles containing cells in a volume of 1.5 L and a total of about 1.0×10⁹ to about 1.0×10¹¹ cells.

Cells were transferred from roller bottles into a perfusion bioreactor for further expansion. The cultivation volume was increased from 5 L to 10 L (final working volume) within following two days. Approximately 24 h after the 10 L working volume was reached, perfusion was started to enhance cell proliferation to reach the target cell density of 0.5×10⁶ to 0.5×10⁷ cells/mL.

Perfusion was continued until the target cell number is reached.

### Example 2. Adherent Cell Expansion and Infection in iCELLis 500+/500 m², Harvest and Bulk Harvest Filtration

Cells from the previous expansion step were diluted into Ex-Cell medium to form an 8 L to 10 L aliquot that was inoculated into the iCELLis 500+/500 m² bioreactor to achieve a cell density of 8000-10,000 cells/cm². Cells were inoculated into the iCELLis 500+/500 m² bioreactor containing 60 L of media (DMEM-9% FBS-2 mM L-GlutaMAX).

Cell proliferation was enhanced by perfusion, started on the second day of cell proliferation in the iCELLis 500+/500 m².

Before the infection, the number of cells was assessed in order to calculate the correct MOI. It is not possible to count cells from the adherent iCELLis 500+/500 m² bioreactor. Therefore, for each batch, the number of cells in the bioreactor was determined theoretically based on the seeding density, expansion time between inoculation and infection, and doubling time of the cells characteristic to HEK293 WCB. Theoretically, when cells have been inoculated at a cell density of 10000 cells/cm², after 110 h cultivation, the cell density would be 140000 cells/cm².

A sufficient amount of a secondary Working Virus Seed Stock (sWVSS) aliquots were thawed in a water bath set at +37° C. Infection of HEK293 cells was performed using a MOI 150-170 vp/cell, or 9-21 infectious particles/cell. Just before the infection, the perfusion was stopped, and the required volume of virus was added to the iCELLis 500+/500 m² system to infect the expanded HEK293 cells. Duration of infection was of 50-60 hours.

Approximately 60 minutes after cells have been infected, the perfusion was restarted. Approximately 30 h post infection, the perfusion media was changed to serum-free media, reducing the concentration of residual FBS at harvest. Perfusion was continued until harvest.

Spent media in the bioreactor was discarded. The bioreactor was refilled with 60 L of fresh DMEM without additives and allowed to equilibrate for 0-30 min until it was in the target temperature range (≥29.5° C.). A primary Benzonase treatment aliquot of approximately 74 U/mL was added to the bioreactor and incubated for 30-35 minutes with temperature control and stirring on. The purpose of the primary Benzonase treatment was to start DNA digestion as soon as the host cell DNA begins to be released from the cells during the lysis step.

Cell lysis was performed with lysis buffer containing 10% m/v polysorbate 20. Lysis was continued for 2 hours±10 min. A secondary Benzonase treatment of approximately 369 U/mL was then added to digest host cell DNA for a further 30-35 minutes prior to inactivation of the Benzonase with a high concentration salt buffer (incubation time 15-20 min at 30-37° C. with constant stirring). After incubation, the content of the bioreactor was collected into a harvest bag. A total of approximately 87 L was recovered. The bioreactor was further rinsed with 120 L of conditioning buffer to recover as many virus particles from the bioreactor bed as possible.

The rinse was pooled with the main harvest material. This pooled harvest material was referred to as “Bulk Harvest” and was further filtered through a 0.5/0.2 μm filter, after which the filters are flushed with total of 20 L of conditioning buffer to obtain a “filtered bulk harvest”.

1. A method for producing a recombinant adenovirus vector, comprising the following steps:
a) inoculating cells in a bioreactor comprising a carrier providing a surface area for adherent cell culture;
b) infecting said inoculated cells with said recombinant adenovirus vector at a multiplicity of infection of about 110 to about 190 virus particles/cell and a duration of infection of 50 to 60 hours;
c) lysing the cells into the bioreactor;
d) performing a harvesting step of the recombinant adenovirus vectors from said lysed cells to obtain a bulk harvest of recombinant adenovirus vectors; and
e) filtering said bulk harvest to obtain a filtered bulk harvest.

2. The method according to claim 1, wherein the cells are HEK293 cells or HEK293T cells, in particular HEK293 cells.

3. The method according to claim 1, wherein the bioreactor comprises a packed-fiber carrier.

4. The method according to claim 1, wherein about 8,000-about 10,000 cells/cm² are seeded into the bioreactor.

5. The method according to claim 1, wherein the infecting step is performed at a MOI of about 150 to about 170 viral particles/cell.

6. The method according to claim 1, wherein lysis is performed using a detergent.

7. The method according to claim 6, wherein the detergent is polysorbate 20.

8. The method according to claim 1, further comprising a two-step endonuclease treatment performed before and after the lysis step.
